(19) United States
(12) Patent Application Publication
(10) Pub. No.: US 2016/0008175 A1
(43) Pub. Date: Jan. 14, 2016

(54) SLEEP MASK SYSTEM

(71) Applicant: Fabrictech 2000, LLC, DBA PureCare, Fairfield, NJ (US)

(72) Inventors: Jeffrey Bergman, Cave Creek, AZ (US); Sean Bergman, Virginia Beach, VA (US)

(21) Appl. No.: 14/750,497

(22) Filed: Jun. 25, 2015

Related U.S. Application Data
(60) Provisional application No. 62/024,110, filed on Jul. 14, 2014.

(51) Int. Cl. A61F 9/04

(52) U.S. Cl. CPC A61F 9/04

(57) ABSTRACT

Sleep mask system comprises a sleep mask having a front and a back, the back having two eye cavities substantially corresponding to the spacing of the user's eyes. A gel pad is removably attachable to the back of the sleep mask, and has eye holes that are aligned with the eye cavities of the sleep mask when attached thereto.

### CROSS-REFERENCE TO RELATED APPLICATION

This application claims the benefit of U.S. Provisional Application Ser. No. 62/024,110, filed Jul. 14, 2014, the entire disclosure of which is incorporated herein by reference.

### TECHNICAL FIELD

This invention relates generally to a sleep mask system comprised of a sleep mask that blocks out light and a gel pad that provides refreshing relief to the face and eyes of the wearer.

### BACKGROUND

There are a wide variety of eye masks on the market today. Most eye masks fall into two general categories of use: (1) to reduce and/or block out light to help with sleep, or (2) to soothe the eyes and/or relieve any number of ailments. Both types of masks are generally shaped to fit over the bridge of the nose and press softly around the eyes. Additionally, most masks come with a strap that extends behind the head to hold the mask in place.

Masks that are designed to block out light are often referred to as sleep masks and are generally made of a soft fabric. They often have some level of padding sewn into the fabric to provide additional comfort and/or provide better light blocking coverage. Most light reducing masks completely cover the eyes and often touch the eyelids when in use. When the fabric comes in contact with the user's eyes, it can cause irritation. As such, some sleep masks are shaped and designed in such a way that there are recesses (cavities) where they cover the eyes. These cavities keep the user's eyes, as well as the eyelashes, from touching the fabric of the mask and allow the user to fully open the user's eyes while the mask is in place.

In contrast, eye masks that are designed to sooth the eyes are generally made of plastic and are filled with some sort of gel-like substance that allows for cooling and/or heating to relieve any number of ailments. Most gel mask manufacturers recommend placing a piece of fabric or tissue between the mask and the eyelid and/or the masks come with a fabric cover to protect the eyelids from possible irritation when in use. Alternatively, the soothing eye masks may have eye cut-outs in the area specifically over the eyes, thus keeping the masks from coming in direct contact with the eyelids.

### SUMMARY

The present invention relates to a sleep mask system that blocks out light while soothing the user's eyes and facial skin during sleep.

In accordance with an embodiment, the sleep mask system is comprised of a sleep mask and a gel pad, the sleep mask of which can be used separately or in combination to provide the user a better sleep experience.

In accordance with an embodiment, the sleep mask fabric is treated with an antimicrobial treatment to inhibit the growth of bacteria, mold and/or mildew and suppress the reproduction of dust mites and/or other allergens.

In accordance with an embodiment, the gel pad is infused with one or more aromatic botanical essential oil extracts such as lavender, neroli, jasmine, and/or jojoba for therapeutic soothing of the areas under and around the user's eyes and facial skin during sleep.

In accordance with an embodiment, the sleep mask system comprises a sleep mask having eye recesses (cavities) where it covers the eyes and a soothing gel pad attached to the back of the sleep mask that has eye hole cutouts that align with the eye recesses in the sleep mask to allow the user to fully open his or her eyes and blink without irritation.

In accordance with an embodiment, the sleep mask has fasteners to allow for ease of attaching and removing the gel pad, as well as to hold the gel pad in place when the sleep mask system is in use.

### BRIEF DESCRIPTION OF THE DRAWINGS

FIG. 1 is a schematic front view of a sleep mask embodiment.

FIG. 2 is a schematic perspective back view of the sleep mask embodiment of FIG. 1.

FIG. 3 is an enlarged back view of the sleep mask of FIG. 2 that has pocket flaps that cover fasteners allowing for ease of attachment and removal of a gel pad from the back of the sleep mask as well as hold the gel pad in place when in use.

FIG. 4 is an enlarged schematic back view of the sleep mask similar to FIG. 3 but showing the pocket flaps folded back to expose the fasteners used to hold the gel pad in place when in use.

FIG. 5 is an enlarged schematic back view of a gel pad embodiment.

FIG. 6 is an enlarged schematic back view of a sleep mask system embodiment comprising the sleep mask shown in FIG. 4 and the gel pad shown in FIG. 5 held in place by fasteners on the back of the sleep mask.

FIG. 7 is an enlarged schematic back view of the sleep mask system of FIG. 6 but showing edges of the gel pad tucked beneath pocket flaps overlying the fasteners.

FIG. 8 is an enlarged transverse section through the sleep mask of FIG. 3 taken on the plane of the line 8-8 thereof.

FIG. 9 is an enlarged transverse section through the sleep mask of FIG. 3 taken on the plane of the line 9-9 thereof.

FIG. 10 is an enlarged transverse section through the sleep mask system of FIG. 7 taken on the plane of the line 10-10 thereof.

### DETAILED DESCRIPTION

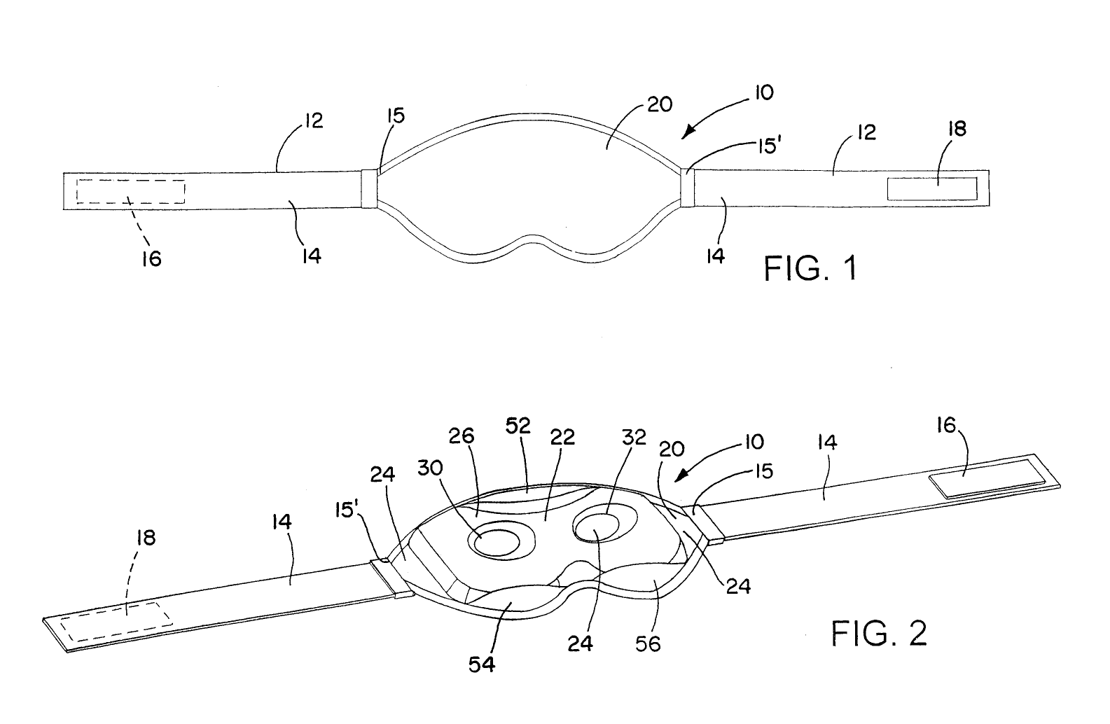

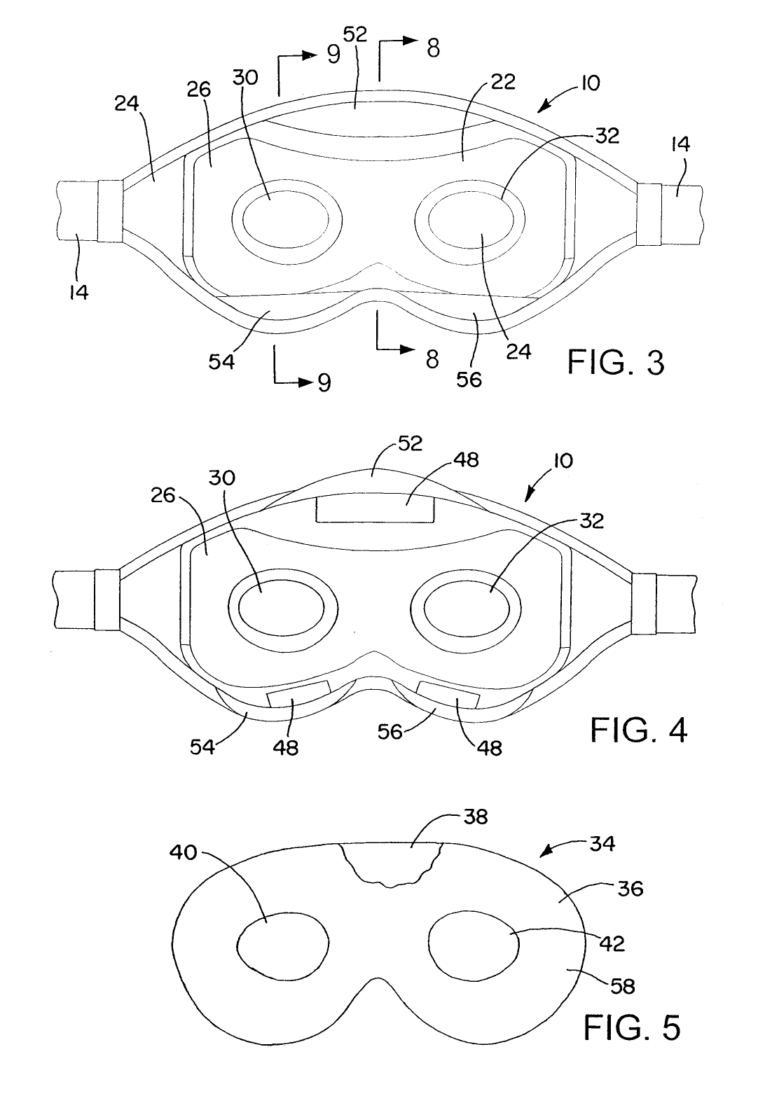

Referring now in more detail to the drawings, and initially to FIGS. 1-4, there is shown a sleep mask 10 in accordance with an embodiment herein made of a suitable soft fabric having a support member 12 attached to opposite sides of the sleep mask that substantially wrap around the user's head to hold the sleep mask in place against the user's face.

In an embodiment, the support member 12 comprises two separate straps 14 affixed to opposite sides 15, 15′ of the sleep mask 10, and fasteners 16, 18 adjacent the outer ends of the straps that are adjustably connectable to one another to allow the straps to be adjusted to the size of the user's head and tightness preference. By way of example, the fasteners 16, 18 may be hook and loop type fasteners (e.g., Velcro® strips). Alternatively, the fasteners may be snaps, buttons or other suitable type fasteners.

In one embodiment, the straps 14 are made of a stretch fabric such as elastic. In another embodiment, the straps 14 are made of a non-stretch fabric with a length adjustment or ties as desired.

In an embodiment, the sleep mask 10 comprises a relatively soft, pliable opaque fabric material 20 having a level of padding 22 made for example of a foam-like or other soft fill material on the back 24 of the sleep mask to provide additional comfort and better light blocking coverage.

In the back 26 of the padding 22 are a pair of eye hole cavities or recesses 30, 32 substantially corresponding to the spacing of a user's eyes. The cavities/recesses 30, 32 are sized to allow for the back of the sleep mask to fit securely/snugly to the user's face around the eyes to block out light without contacting the user's eyes, thus allowing the user to blink without irritation.

Although the size of the eye recesses/cavities 30, 32 may vary within certain limits, in an embodiment, the eye cavities/recesses have a width of between approximately 3 cm. and approximately 6 cm. and a height of between approximately 2 cm. and approximately 5 cm. Also the thickness of the padding 22 must be sufficient to accommodate the depth of the eye recesses/cavities, which may range from between approximately 0.8 cm. and approximately 2.5 cm., to allow the user to fully open the user's eyes and blink without irritation.

In an embodiment, the fabric 20 of the eye mask 10 including the fabric 20 covering the padding 22 is treated with an anti-microbial treatment to inhibit the growth of bacteria, mold and/or mildew and suppress the reproduction of dust mites and/or other allergens.

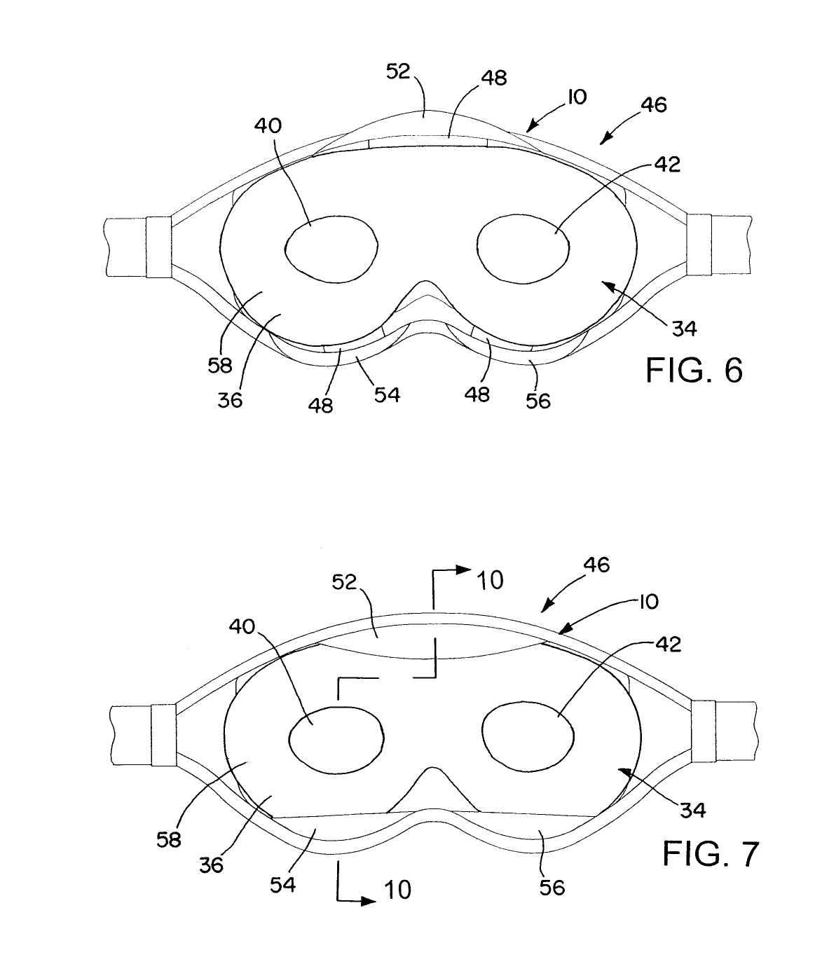

In an embodiment, a gel pad is removably attachable to the back of the sleep mask 10 to provide a sleep mask system in accordance with an embodiment herein as described hereafter. A gel pad embodiment 34 is shown in FIG. 5 that comprises a relatively thin gel layer 36 made of a suitable stable gel material such as thermoplastic rubber (TPR) and a fabric backing 38 for use in releasably attaching the gel pad to the sleep mask. The gel pad 34 including the gel layer 36 and the fabric backing 38 have eye hole cutouts 40, 42 that are aligned with the eye recesses/cavities 30, 32 in the sleep mask when attached thereto to provide a sleep mask system 46 (shown in FIGS. 6, 7 and 10) that allows the user to blink without irritation of the eyes.

In an embodiment, the gel pad layer 36 is infused with one or more aromatic botanical essential oil extracts such as lavender, neroli, jasmine, and/or jojoba during the molding process for therapeutic soothing of the areas under and around the user's eyes and facial skin.

In an embodiment, the gel pad 34 contours to the curvature of the user's cheekbones and brow, helping to alleviate muscle tension around the eyes. Also in an embodiment, the gel pad is substantially the same size as the sleep mask padding 22 except where the padding has one or more cutouts for mounting one or more suitable fasteners 48 for removably attaching the gel pad to the back of the sleep mask.

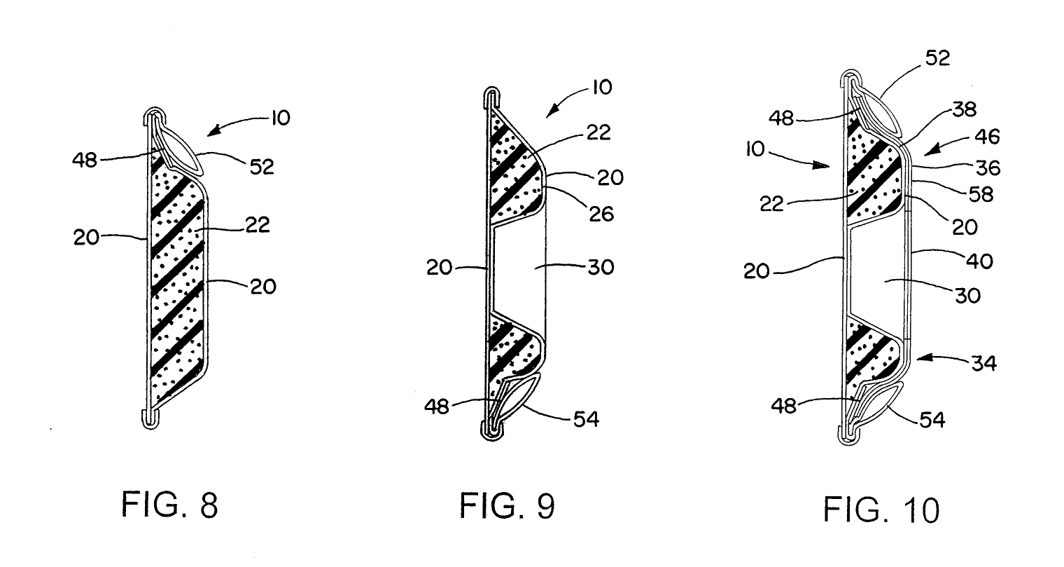

In an embodiment, hook and loop type fasteners 48 (shown in FIGS. 4, 6 and 8-10) (e.g.,—Velcro® strips) or other type fasteners such as snaps, buttons, tape or the like are engaged by the fabric backing of the gel pad 34 to hold the gel pad in place during use and permit easy removal of the gel pad for replacement when no longer effective or when finished using the gel pad. If hook and loop type fasteners are used, the fabric backing is desirably made of a loop type fabric such as flannel for engagement by fastener hooks on the back of the sleep mask.

In an embodiment, fabric flap covers 52, 54 and 56 (see FIGS. 2-4 and 6-10) overlie the fasteners 48 to protect against the fasteners touching the user's facial skin if the sleep mask is used without the gel pad. Also in an embodiment, three fasteners 48 are located on the back of the sleep mask 10, one above the user's nose and the other two near the user's cheekbones to allow for ease of attachment and removal of the gel pad as well as to hold the gel pad in place when in use. However, more or fewer fasteners may be provided at the same or different locations if desired.

In an embodiment, the fabric flap covers are treated with the same anti-microbial treatment as the rest of the eye mask fabric.

In an embodiment, one or more gel pads 34 may be stored in a resealable silver packet (not shown) with a protective film applied to the exposed surface 58 of the gel pads to extend the useful life of the gel pads.

In an embodiment, to attach a gel pad 34 to the sleep mask 10 to provide a sleep mask system 46, the sleep mask is desirably laid flat with the back facing up. Then respective edges of the gel pad (e.g., after removal of the gel pad from the resealable packet and peeling of the protective film away from the gel pad) are tucked beneath the protective flaps 52, 54, 56 with the exposed surface 58 of the gel pad facing rearwardly and the fabric backing 38 facing forwardly and secured in place by engagement of the fabric backing with the fasteners 48. Thereafter the sleep mask system may be fitted over the bridge of the user's nose and pressed softly around the eyes and adjusted as needed for proper fit. When the exposed surface 58 of the gel pad touches the face, the essential oils will come out of the gel pad slowly and gently soothe the areas under and around the user's eyes and facial skin.

When finished using the gel pad, the gel pad is desirably removed from the sleep mask and a protective film is reapplied to the exposed surface 58 of the gel pad and stored in a resealable packet for extending the useful life of the gel pad.

Although the invention has been shown and described with respect to certain embodiments, it is obvious that equivalent alterations and modifications will occur to others skilled in the art upon reading and understanding of the specification. In particular, with regard to the various functions performed by the above described components, the terms (including any reference to a “means”) used to describe such components are intended to correspond, unless otherwise indicated, to any component which performs the specified function of the described component (e.g., that is functionally equivalent), even though not structurally equivalent to the disclosed component which performs the function of the herein illustrated exemplary embodiments of the invention. In addition, while a particular feature of the invention may have been disclosed with respect to a particular embodiment, such feature may be combined with one or more other features as may be desired or advantageous for any given or particular application.

What is claimed is:

1. A sleep mask system comprising:
a sleep mask having a front and a back, the back having two eye cavities substantially corresponding to the spacing of a user's eyes;
a gel pad removably attachable to the back of the sleep mask, the gel pad having eye holes that are aligned with the eye cavities of the sleep mask when attached thereto; and
a support member attached to opposite sides of the sleep mask that substantially wraps around a user's head to hold the sleep mask system in place against a user's face.

2. The mask system of claim 1, wherein the back of the sleep mask has a level of padding containing the eye cavities, the padding having a thickness that is sufficient to accommodate a depth of the eye cavities that is sufficient to allow the user's eyes to fully open and blink without irritation.

3. The mask system of claim 1 wherein the sleep mask is treated with an antimicrobial substance known to suppress the reproduction of dust mites or allergens and inhibit the growth of bacteria, mold or mildew.

4. The mask system of claim 1 wherein the gel pad is infused with one or more botanical essential oil extracts for skin and eye benefit.

5. The mask system of claim 4 wherein the gel pad has a fabric backing that faces the back of the sleep mask when attached thereto, and an exposed surface that comes into contact with a user's skin when the sleep mask is fitted to the user's face.

6. The mask system of claim 1 further comprising a fastening device for removably affixing the gel pad to the back of the sleep mask.

7. The mask system of claim 6 wherein the fastening device comprises fasteners on the back of the sleep mask.

8. The mask system of claim 7 wherein the fasteners are located on the back of the sleep mask above a user's nose and near a user's cheekbones when the sleep mask is fitted to the user's face to allow for ease of attaching and removing the gel pad as well as to hold the gel pad in place when in use.

9. The mask of claim 6 wherein flap covers overlie the fasteners to protect against the fasteners touching a user's skin if the sleep mask is used without the gel pad when the sleep mask is fitted to the user's face.

10. The mask system of claim 1 wherein the support member is comprised of two separate straps affixed to opposite sides of the sleep mask, and fasteners on the straps that allow the straps to be adjusted to the user's head size and tightness preference.

11. The mask system of claim 10 wherein the straps are made of a stretch fabric.

12. A sleep mask system comprising:
a sleep mask having a front and a back, the back having two eye cavities substantially corresponding to the spacing of a user's eyes;
a gel pad having eye holes that are aligned with the eye cavities of the sleep mask when attached thereto; and
a fastening device for removably attaching the gel pad to the back of the sleep mask.

13. The mask system of claim 12 wherein the gel pad has a fabric backing that faces the back of the sleep mask when attached thereto, and an exposed surface that comes into contact with a user's skin when the sleep mask is fitted to a user's face.

14. The mask system of claim 13 wherein the fastening device comprises one or more fasteners for removably attaching the fabric backing of the gel pad to the back of the sleep mask.

15. The mask system of claim 14 wherein the fasteners are on the back of the sleep mask.

16. The mask system of claim 15 wherein the fasteners are located on the back of the sleep mask above a user's nose and near a user's cheekbones when the sleep mask is fitted to the user's face to allow for ease of attaching and removing the gel pad as well as to hold the gel pad in place when in use.

17. The mask system of claim 15 wherein flap covers overlie the fasteners to protect against the fasteners touching a user's skin if the sleep mask is used without the gel pad.

18. The mask system of claim 12 wherein the gel pad is infused with one or more botanical essential oil extracts for skin and eye benefit.

19. The mask system of claim 12 wherein the sleep mask is treated with an antimicrobial substance to suppress the reproduction of dust mites or allergens and inhibit the growth of bacteria, mold or mildew.
